(19) United States
(12) Patent Application Publication
(10) Pub. No.: US 2003/0068349 A1
(43) Pub. Date: Apr. 10, 2003

(54) Use of 5'-deoxy-5'-methylthioadenosine in skin cosmetic compositions

(76) Inventors: Axel Jentzsch, Mannheim (DE); Karl Kolter, Limburgerhof (DE); Karin Sperling, Neustadt (DE)

Correspondence Address: Hebert B. Keil, KEIL & WEINKAUF, 1350 Connecticut Ave., N.W., Washington, DC, 20036, US

(21) Appl. No.: 10/187,658

(22) Filed: Jul. 3, 2002

(30) Foreign Application Priority Data
Jul. 4, 2001 (DE) 10132338.7

(51) Int. Cl. A61K 7/00; A61K 31/52

(52) U.S. Cl. 424/401; 514/263.23

(57) ABSTRACT

The present invention relates to the use of 5′-deoxy-5′-methylthioadenosine in skin cosmetic compositions for improving the appearance of the skin, to such compositions and to a method for the cosmetic treatment of the skin.

The present invention relates to the use of 5′-deoxy-5′-methylthioadenosine in skin cosmetic compositions for improving the appearance of the skin, to such compositions and to a method for the cosmetic treatment of the skin.

The human skin is subject to changes which are not types of skin disorders which require treatment, but which nevertheless are regarded as being in need of improvement by those affected. These include, for example, aging processes, it being possible to make a distinction between intrinsic processes (chronoaging) and exogenous factors. In addition, for example, temporary or permanent changes in the appearance of the skin also arise, such as greasy or dry skin and others whose exact causes, and factors which influence them, are often not entirely understood.

Exogenous factors include, for example, sunlight or artificial radiation sources having a comparable spectrum, and compounds which can form as a result of the radiation, such as undefined reactive, e.g. free-radical or ionic, photoproducts. These factors also include, however, harmful or reactive compounds, such as ozone, free radicals, for example the hydroxyl radical, singlet oxygen and other reactive oxygen or nitrogen compounds, cigarette smoke, natural and synthetic toxins, and others which interfere with the natural physiology or morphology of the skin. The effect of these factors may result, for example, in direct damage to the DNA of the skin cells, and to the collagen, elastin and glycosaminoglycan molecules of the extracellular matrix which are responsible for the strength of the skin. In addition, the harmful influences may, however, also result in damage to the cells of the skin itself. Consequently, the regeneration ability of the skin, for example, is reduced. A further consequence which may result is inflammatory reactions, where, inter alia, immunoregulatory compounds, such as interleukins, prostaglandins and histamines, play a role.

The consequences of aging are thinning of the skin, weaker meshing of the epidermis and dermis, reduction in cell number and in supplying blood vessels. The aging processes lead to the formation of fine lines and wrinkles, the skin becomes leathery, yellowish and sagging, and pigment disorders arise.

It is known that exposure to sunlight leads to a large number of undesired effects in the skin: erythemae, photosensitization and immunological changes are examples of primarily acute reactions, while photoaging and carcinogenesis represent long-term effects. In addition, it is known that, following irradiation of the skin, matrix-degrading enzymes are induced and that this induction can be reduced by low molecular weight compounds, such as, for example, retinoic acids. A further effect of UV radiation is the appearance of sunburn cells in the skin. As a result, necrotic processes may arise, which induce or intensify an inflammatory reaction.

The same factors also act upon hair, where damage may also arise. The hair becomes brittle, less elastic and dull; the surface structure of the hair becomes damaged.

The properties of cosmetic compositions are therefore subject to a large number of requirements. For example, they should, inter alia, have a free-radical scavenging action, an antiinflammatory action or a moisturizing action, they should prevent or reduce the activity of matrix-degrading enzymes and/or regulate the synthesis of new collagen, elastin and/or proteoglycans.

WO-A-97/30713 describes compositions for inhibiting irreversible cell changes as a result of stress, in particular lack of oxygen, as arises, for example, ex vivo during the storage of cells, e.g. of organs for transplantation, and in vivo in the case of diverse operative interventions. These compositions comprise at least one physiologically compatible purine derivative, preferably a purine riboside. As well as compositions for the storage of cells, food compositions are also described. Topical applications of these compositions are, however, not described.

EP-A-0 526 866 describes the use of adenosine compounds, such as 5′-deoxy-5′-methylthioadenosine for the preparation of pharmaceutical compositions for the treatment of ischamia.

EP-A-0 352 609 relates to the use of adenosine derivatives, such as 5′-deoxy-5′-methylthioadenosine, for the preparation of pharmaceutical compositions with immunostimulating action.

EP-A-0 184 248 describes salts of 5′-deoxy-5′-methylthioadenosine with long-chain sulfonic acids which have a central and peripheral vasodilatory action, an antiblood-platelet clumping action, an antiinflammatory action, an analgesic action and an antipyretic action.

U.S. Pat. No. 4,373,097 describes a process for the preparation of adenosine derivatives such as 5′-deoxy-5′-methylthioadenosine and their antiinflammatory, analgesic and antipyretic action.

R. Tritapepe and C. Pozzi described, in Acta Therapeutica 15, 1989, pages 299-311, the use of 5′-methylthioadenosine for the topical treatment of venal disorders, such as superficial thrombophlebitis, acute venous thrombosis, varicose veins and varicose ulcers. The use of 5′-methylthioadenosine in cosmetics is not described.

G. Pinto et al. describe, in Drug Invest 4 (3), pages 205-214, 1992, the topical application of 5′-methylthioadenosine for the treatment of the symptoms of chronic venous insufficiency, of hemorrhoids and superficial phlebitis. A use in cosmetics is again not described.

M. A. Cerri et al. describe, in the European Journal of Pharmacology, 232, 1993, pages 291-294, the inhibition of cytokine production and of endothelial expression of adhesion antigens by 5′-methylthioadenosine.

EP-A-0 191 561 describes a composition against caries or parodentosis which comprises, as active component, adenosine or an adenosine derivative, such as 5′-deoxy-5′-methylthioadenosine.

EP-A-0 387 757 describes the use of 5′-deoxy-5′-methylthioadenosine for the preparation of oral and topical pharmaceutical compositions for stimulating hair growth in people with alopecia.

EP-A-0 387 756 relates to the use of 5′-deoxy-5′-methylthioadenosine in pharmaceutical compositions which is suitable for reducing excessive sebaceous gland secretion on the head and associated formation of dandruff and skin itchiness. These compositions are suitable for oral and topical application.

WO-A-94/14428 describes a method of reducing hair growth in mammals, where a composition with an effective amount of a sulfhydryl-active compound is applied to the skin. This sulfhydryl-active compound may be 5′-deoxy-5′-methylthioadenosine.

WO 98/15276 describes a method of regulating melanin production in the skin or the hair, by applying an effective amount of a pharmaceutical formulation which comprises a regulating agent thereto. This regulating agent may be an adenosine-2 receptor antagonist, such as 5′-deoxy-5′-methylthioadenosine.

It is an object of the present invention to provide a method for the cosmetic treatment of the skin and/or the hair which is suitable for improving the appearance of the skin. The method should, in particular, be suitable for the treatment of age-induced cosmetically undesired changes in the appearance of the skin. In addition, the intention is also to prevent skin and hair damage and/or undesired changes in the appearance of the skin as far as possible.

Surprisingly, we have found that this object is achieved by using 5′-deoxy-5′-methylthioadenosine in skin cosmetic compositions.

For the purposes of the present application, “improvement in the appearance of the skin” is understood as meaning any positive cosmetic change, or also any cosmetic change regarded as positive by the user, in the appearance of the skin. The use according to the invention for the treatment of cosmetically undesired changes in the appearance of the skin relates to the use brought about essentially out of esthetic considerations on the part of the user. It does not relate, or only relates to a minor degree, to a use brought about essentially out of medical-therapeutic considerations. The use according to the invention does not cover the stimulation or reduction in hair growth, nor use in topical compositions for accelerating skin tanning and for protecting the skin against ultraviolet rays (light protection compositions) in the sense of a physical barrier. The use according to the invention, however, covers the cosmetic treatment of skin changes which may, inter alia, be (simultaneously) caused by the action of light.

The term “5′-deoxy-5′-methylthioadenosine” refers for the purposes of the present invention to compounds of the formula I

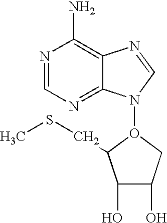

The compounds of the formula I are also referred to as 5′-methylthioadenosine, MTA, 7-[tetrahydro-3,4-dihydroxy-5-(methylthiomethyl)-2-furyl]adenine and vitamin L₂.

The compounds of the formula I can be used in the form of pure optical isomers or as mixtures.

The use according to the invention also includes cosmetically acceptable salts. Preferred cosmetically acceptable salts of 5′-deoxy-5′-methylthioadenosine are acid addition salts.

The acid addition salts include salts with inorganic acids, for example phosphoric acid, or with carboxylic acids, such as mono- or polyunsaturated C₆-C₃₀-monocarboxylic acids, such as caprylic acid, pelargonic acid, capric acid, undecanoic acid, lauric acid, tridecanoic acid, myristic acid, pentadecanoic acid, palmitic acid, margaric acid, stearic acid, nonadecanoic acid, arachidic acid, behenic acid, lignoceric acid, cerotinic acid, melissic acid, palmetoleic acid, oleic acid, linoleic acid, linolenic acid, elaostearic acid, and hydroxycarboxylic acids, such as lactic acid, polyhydric carboxylic acids, such as citric acid and mixtures thereof.

5′-Deoxy-5′-methylthioadenosine and the salts thereof are suitable in an advantageous manner for the prevention and treatment of damage to keratinic surfaces, i.e. skin and skin appendages, such as, for example, hair, nails etc., of individuals, preferably mammals, in particular humans, useful animals or domestic animals. The use takes place here in the form of cosmetic compositions such as body care compositions, decorative cosmetics etc., which do not generally require a prescription. This is to be differentiated from the application in the form of dermatological agents, which is understood as meaning medicaments for the therapy of skin disorders (dermatoses).

Cosmetic preparations based on 5′-deoxy-5′-methylthioadenosine and/or salts thereof offer effective protection against oxidative processes, against processes caused by radiation or reactive compounds, against damage caused directly or indirectly by such processes. They are advantageously suitable for the treatment of cosmetic or dermatological changes to skin and hair, such as e.g. skin aging, loss of skin moisture, loss of skin elasticity, the formation of wrinkles or lines or of pigment disorders or age spots.

In addition, the present invention relates to the use for the cosmetic treatment or prevention of undesired changes in the appearance of the skin, such as acne or greasy skin.

5′-Deoxy-5′-methylthioadenosine is particularly preferably suitable for use in skin cosmetic compositions for the treatment of a skin appearance changed as a result of aging. MTA serves here e.g. for the rejuvenation and/or revitalization of the skin. Advantageously, energizing effects can also usually be observed. In particular, 5′-deoxy-5′-methylthioadenosine has a positive effect on the function of the mitochondria. Surprisingly, in this way, not only is oxidative damage to the skin prevented, but existing damage can, at least partially, be repaired. In the case of the use according to the invention, improvements in the moisture value and/or the elasticity of the skin are recorded. The use of 5′-deoxy-5′-methylthioadenosine also increases the further synthesis of collagen and/or elastin. This generally leads to an at least partial smoothing of lines, and the complexion looks more radiant and fresher. As a rule, said advantageous effects are also accompanied by a positive subjective sensation of having overall “youthful” skin.

Formulations based on 5′-deoxy-5′-methylthioadenosine are also advantageously suitable for the treatment, care and cleansing of the skin or of the hair and can be used as a make-up product in cosmetics. The skin cosmetic composition in which 5′-deoxy-5′-methylthioadenosine is used generally comprise at least one cosmetically acceptable carrier. Suitable carriers are described below. They preferably comprise 0.001% by weight to 30% by weight of the active ingredient. The composition depends here, for example, on the penetration properties of the active substance through the stratum corneum and its ability to form a depot in the skin.

According to a preferred embodiment, the application according to the invention of 5′-deoxy-5′-methylthioadenosine and/or salts thereof advantageously takes place by regular application, e.g. in the form of a cosmetic preparation, over a period of time. This depends on the desired aim, i.e. the period of time can extend over the lifetime of the user, preferably over a period up to three months, particularly preferably over a week to two months if the aim is to build up a depot in the skin. For an aftersun application, the application duration for the purposes of the invention is the single application, but preferably a period of at least one day, particularly preferably over three days to three months, especially preferably over one to two weeks.

For the purposes of the invention, it is advisable to topically apply the cosmetic preparation of 5′-deoxy-5′-methylthioadenosine in an amount of from 0.1 μg/cm² to 2 mg/cm², between once per week and 4 to 5 times daily, preferably 3 times per week to 3 times daily, particularly preferably once to twice daily. Amounts of active ingredient and proportions are based on the active ingredient, meaning that for salts and derivatives it is necessary to make the appropriate conversion.

Administration forms of 5′-deoxy-5′-methylthioadenosine intended for aftersun applications, and optionally additional active ingredients, advantageously have penetration properties which permit rapid penetration of the substance into the skin. On the other hand, for applications with “preconditioning” character, rapid penetration is usually unimportant, whereas the ability to build up a depot in the skin is advantageous.

Surprisingly, in the case of the use according to the invention, effective treatment and also prevention of prematurely aged skin (e.g. wrinkles, age spots, telangiectases, pigment disorders) and/or prematurely aged skin appendages, radiation-induced skin damage or radiation-induced adverse changes in the skin and/or the skin appendages, environmentally induced (ozone, free radicals, singlet oxygen, reactive oxygen or nitrogen compounds, cigarette smoke, toxins) skin damage or environmentally induced adverse changes in the skin and/or the skin appendages, light-sensitive, inflammatory, erythematous, allergic or autoimmune-reactive changes in the skin and/or the skin appendages (in particular acne, greasy or dry skin, keratoses, rosaceae, dermatoses, atopic eczema, seborrhoic eczema, photodermatoses, polymorphous light dermatoses), deficient, sensitive or hypoactive states of the skin and/or the skin appendages, itching, dry skin states and horny layer barrier disorders and/or hair loss and reduced hair growth is possible.

The novel cosmetic application of 5′-deoxy-5′-methylthioadenosine and/or salts thereof also serves, however, in a surprising and unforeseen manner, for calming sensitive and irritated skin. It furthers serves to regulate the synthesis of collagen, hyaluronic acid and elastin, to stimulate DNA synthesis, in particular in the case of deficient or hypoactive skin states, to regulate the transcription and translation of matrix-degrading enzymes, in particular matrix metalloproteinases, to increase cell renewal and regeneration of the skin, to increase the skin's own protective and repair mechanisms for DNA, lipids and/or proteins. 5′-Deoxy-5′-methylthioadenosine can also be used in cosmetic compositions for after-treatment in surgical interventions, in particular to counteract skin irritations and to promote the regeneration processes of injured skin.

For the use according to the invention, the cosmetic preparations are applied to the skin and/or the hair in a sufficient amount in the manner customary for cosmetics.

For example, 5′-deoxy-5′-methylthioadenosine or a salt thereof is used in cosmetic compositions for cleansing the skin, such as bath soaps, toilet soaps, cured soaps, transparent soaps, luxury soaps, deodorizing soaps, cream soaps, baby soaps, skin protection soaps, abrasive soaps, syndets, liquid soaps, pasty soaps, soft soaps, washing pastes, liquid washing, showering and bath preparations, e.g. washing lotions, shower preparations, shower gels, foam baths, cream foam baths, oil baths, bath extracts, scrub preparations, in-situ products, shaving foams, shaving lotions, shaving creams.

5′-Deoxy-5′-methylthioadenosine is also suitable for skin cosmetic preparations, such as W/O or O/W skin and body creams, day and night creams, eye creams, aftersun products, hand care products, face creams, multiple emulsions, gelees, microemulsions, liposome preparations, niosome preparations, antiwrinkle creams, face oils, lipogels, sport gels, moisturizing creams, bleaching creams, vitamin creams, skin lotions, care lotions, ampoules, aftershave lotions, preshaves, moisturizing lotions, cellulite creams, depigmentation compositions, massage preparations, body powders, face tonics, face masks, deodorants, antiperspirants, nose strips, antiacne compositions, repellents, shaving compositions, personal hygiene compositions, foot care compositions, baby care compositions and others.

In addition, 5′-deoxy-5′-methylthioadenosine or a salt thereof can be used in cosmetic compositions for hair care, such as hair treatments, hair lotions, hair rinses, hair emulsions, split-end fluids, neutralizing agents for permanent waves, hot-oil treatment preparations, conditioners, setting lotions, shampoos, hair tints and colorants, hairsprays, blow-drying lotions, blow-drying setting compositions, shine sprays, hair brillantines, hairstyling products, hair tonics and others.

5′-Deoxy-5′-methylthioadenosine or a salt thereof are also suitable for use in cosmetic preparations for decorative cosmetics, for example as make-up, powders, blushers, eye shadows, kohl pencils, eyeliners, eye foundation creams, lipsticks, eyebrow pencils, contour pencils, concealing sticks, stage make-up, mascara, eyelash tinting and coloring products, make-up removal products and others.

The cosmetic or hygienic preparations can, depending on the field of use, be prepared as spray (pump spray or aerosol), foam, gel, gel spray, lotion, cream, mousse, ointment, suspensions or powders.

It is also advantageous to apply 5′-deoxy-5′-methylthioadenosine or a salt thereof, optionally with further active ingredients, in encapsulated form, e.g. as cellulose encapsulation, in gelatin, with polyamides, in niosomes, wax matrices, with cyclodextrins or liposomally encapsulated.

The preparations can comprise cosmetic auxiliaries as are customarily used in such preparations, e.g. preservatives, bactericides, perfumes, antifoams, dyes, pigments, thickeners, surface-active substances, emulsifiers, emollients, finishing agents, fats, oils, waxes or other customary constituents of a cosmetic or dermatological formulation, such as alcohols, polyols, polymers, foam stabilizers, solubilizers, electrolytes, organic acids, organic solvents or silicone derivatives.

In addition to said active ingredients, the preparations can comprise further compounds which have an antioxidative, free-radical scavenging, skin moisturizing or moisture-retaining, antierythematous, antiinflammatory or antiallergic action, in order to supplement or enhance the action thereof. In particular, these compounds can be chosen from the group of vitamins, plant extracts, α- and β-hydroxy acids, ceramides, antiinflammatory, antimicrobial and UV-filtering substances, and derivatives and mixture thereof.

The antioxidants are advantageously chosen from amino acids (e.g. glycine, histidine, tryrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glycerol esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate of benzoin resin, rutinic acid and derivatives thereof, butylhydroxytoluene, butylhydroxyanisole, nordihydroguaiacic acid, nordihydroguaiaretic acid, trihydroxybutyrophenone, uric acid and derivatives thereof, mannose and derivatives thereof, sesame oil, sesamolin, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of said active ingredients which are suitable according to the invention.

The antioxidants can be used alone or in the form of mixtures. The amount of antioxidants used in the preparations, alone or in combination, is preferably 0.001 to 30% by weight, based on the total weight of the preparation.

In addition, the preparations preferably comprise substances which absorb UV radiation in the UV-B and/or UV-A region. Suitable UV filters are e.g. 2,4,6-triaryl-1,3,5-triazines in which the aryl groups can in each case carry at least one substituent which is preferably chosen from hydroxyl, alkoxy, specifically methoxy, alkoxycarbonyl, specifically methoxycarbonyl and ethoxycarbonyl, and mixtures thereof. Also suitable are 4-aminobenzoic esters, where the amino group can optionally be alkylated or alkoxylated. These include, for example, isooctyl N,N-dimethyl-4-aminobenzoate. Also suitable are 2-hydroxybenzoic esters, such as, for example, the isooctyl ester. Further suitable UV filters are 2,4,6-trianilino-(o-carbo-2-ethylhexyl-1′-oxy)-1,3,5-triazine, 3-imidazol-4-ylacrylic acid and its ethyl ester, menthyl o-aminobenzoate, glyceryl p-aminobenzoate, 2,2′-dihydroxy-4-methoxybenzophenone (dioxybenzone), 2-hydroxy-4-methoxy-4-methyl-benzophenone (Mexenon®), triethanolamine salicylate, dimethoxy-phenylglyoxalic acid, 3-(4′-sulfo)benzylidenebornan-2-one and its salts, 2,2′,4,4′-tetrahydroxybenzophenone, 2,2′-methylenebis-[6(2H-benzotriazol-2-yl-4-(1,1,3,3,-tetramethylbutyl)phenol], 2,2′-(1,4-phenylene)-bis-1H-benzimidazole-4,6-disulfonic acid and its Na salt, 2,4-bis[4-(2-ethylhexyloxy)-2-hydroxy]phenyl-6-(4-methoxyphenyl)-(1,3,5)-triazine, 3-(4-methylbenzylidene)-camphor, polyethoxyethyl 4-bis(polyethoxy)paraaminobenzoate, 2,4-dihydroxybenzophenone and/or 2,2′-dihydroxy-4,4′-dimethoxy-benzophenone-5,5′-disodium sulfonate.

The list of said UV filters which can be used in combination with the novel active ingredient combinations is not of course intended to be limiting.

The total amount of filter substances is usually 0.1% by weight to 30% by weight, preferably 0.5 to 15% by weight, in particular 1 to 10% by weight, based on the total weight of the preparations, in order to provide cosmetic preparations which protect the skin from the entire range of ultraviolet radiation.

If a lipid phase is used, then this is preferably chosen from mineral oils, mineral waxes, branched and/or unbranched hydrocarbons and hydrocarbon waxes, triglycerides of saturated and/or unsaturated, branched and/or unbranched C₈-C₂₄-alkanecarboxylic acids; synthetic, semisynthetic or natural oils, such as olive oil, palm oil, almond oil or mixtures; oils, fats or waxes; esters of saturated and/or unsaturated, branched and/or unbranched C₃-C₃₀-alkanecarboxylic acids and saturated and/or unsaturated, branched and/or unbranched C₃-C₃₀-alcohols, esters of aromatic carboxylic acids and saturated and/or unsaturated, branched and/or unbranched C₃-C₃₀-alcohols, for example isopropyl myristate, isopropyl stearate, hexyldecyl stearate, oleyl oleate; synthetic, semisynthetic and natural mixtures of the abovementioned esters, such as jojoba oil, alkyl benzoates or silicone oils, such as, for example, cyclomethicone, dimethylpolysiloxane, diethylpolysiloxane, octamethylcyclotetra-siloxane and mixtures thereof or dialkyl ethers.

If an aqueous phase is used, then this optionally additionally comprises a water-miscible solvent, such as C₁-C₁₀-, preferably C₁-C₅-alcohols, -diols or -polyols, and ethers thereof, preferably ethanol, isopropanol, propylene glycol, glycerol, ethylene glycol monoethyl ether etc.

Suitable emulsifiers are preferably known W/O and o/W emulsifiers, such as polyglycerol esters, sorbitan esters or partially esterified glycerides.

Suitable solubilizers are, in particular, ethoxylated sorbitan esters, ethoxylated lanolin alcohols and ethoxylated castor oil.

Customary native and synthetic thickeners or gel formers in the formulations are crosslinked polyacrylic acids and derivatives thereof, polysaccharides, such as xanthan gum or alginates, carboxymethylcellulose or hydroxycarboxymethylcellulose, hydrocolloids, such as gum arabic or montmorillonite minerals, such as bentonites or fatty alcohols, polyvinyl alcohol and polyvinylpyrrolidone.

Suitable propellants for aerosols are the customary propellants, for example propane, butane, pentane, dimethyl ether and others.

The invention further provides a skin cosmetic composition, except compositions for accelerating tanning and light protection resulting therefrom, comprising

I) 5′-deoxy-5′-methylthioadenosine and/or a cosmetically acceptable salt thereof,

II) optionally at least one further cosmetic active ingredient, and

III)a cosmetically acceptable carrier.

An excluded light protection composition is understood here as meaning a composition which is applied directly before the skin and/or the hair are exposed to light in order to avoid direct damage of the skin and/or the hair, as is caused, in particular, by the UV-B content in sunlight.

The compositions preferably comprise the components I in an amount of from 0.001 to 30% by weight, in particular 0.01 to 25% by weight, based on the total weight of the composition.

With regard to suitable components II) and III), reference is made to that stated previously.

The present invention further provides compositions in the form of a commercial pack with at least one composition based on 5′-deoxy-5′-methylthioadenosine, optionally together with instructions for cosmetic application.

It will of course be appreciated that commercial packs according to the invention may also comprise further preparations, in particular formulations containing active ingredients, and comprehensive instructions which go beyond the abovementioned contents.

The invention further provides a method for the cosmetic treatment of the skin, in which a cosmetic preparation comprising 5′-deoxy-5′-methylthioadenosine and/or a cosmetically acceptable salt thereof is applied to the skin in an amount effective for improving the appearance of the skin.

The invention is illustrated in more detail by reference to the nonlimiting examples below.

### EXAMPLES OF COSMETIC PREPARATIONS

| Formulation type | Area of use | Example No. |
|---|---|---|
| O/W emulsion | Soft skin lotion | 1 |
| W/O emulsion | Protective hand cream | 2 |
| | Suncare lotion | 3 |
| Multiple emulsion | W/O/W emulsion | 4 |
| Microemulsion | Microemulsion | 5 |
| Hydrophilic gel | Liposome gel | 6 |
| Lipophilic gel | Blunted oil gel | 7 |
| | Oil gel | 8 |
| Stick formulation | Sun protection lipstick | 9 |
| | balm | |
| Aqueous cosmetics | Cooling body splash | 10 |
| Decorative cosmetics | Make-up | 11 |
| | Liquid make-up | 12 |
| Oils | Suncare oil | 13 |
| Body cleansers | Deep face cleanser | 14 |
| Rinse-out hair | Conditioners | 15 |
| after treatments | | |
| Leave-in hair after- | Hair wax | 16 |
| treatments | Antidandruff hair tonic | 17 |
| Aerosol | Foot deodorant spray | 18 |
| | Hairspray | 19 |

### EXAMPLES OF COSMETIC PREPARATIONS

### Example 1

### Soft Skin Fluid

| | % by wt. |
|---|---|
| Ceteareth-6 (reaction product of fatty alcohol | 2.50 |
| and ethylene oxide) and stearyl alcohol | |
| Ceteareth-25 (reaction product of fatty alcohol | 2.50 |
| and ethylene oxide) | |
| Hydrogenated coconut fatty glycerides | 1.50 |
| Polyethylene glycol (40 EO units)- | 3.00 |
| dodecyl glycol copolymer | |
| Dimethicone | 3.00 |
| Phenethyldimethicone | 2.00 |
| Cyclomethicone | 1.00 |
| Cetearyl octanoate | 5.00 |
| Avocado oil | 1.00 |
| Sweet almond oil | 2.00 |
| Wheatgerm oil | 0.80 |
| Panthenol USP | 1.00 |
| Phytantriol | 0.20 |
| Tocopheryl acetate | 0.30 |
| Propylene glycol | 5.00 |
| Perfume | q.s. |
| Preservatives | q.s. |
| 5′-Deoxy-5′-methylthioadenosine | q.s. |
| Water | 69.20 |

### Example 2

### Protective Hand Cream

| | % by wt. |
|---|---|
| Cetearyl alcohol | 1.00 |
| Glyceryl stearate | 1.50 |
| Stearyl alcohol | 1.50 |
| Cetyl palmitate | 2.00 |
| Tocopheryl acetate | 0.50 |
| Dimethicone | 8.00 |
| Ceteareth-6 (reaction product of fatty alcohol | 3.00 |
| and ethylene oxide) and stearyl alcohol | |
| Octyl methoxycinnamate | 5.00 |
| Propylene glycol | 8.00 |
| Panthenol | 1.00 |
| Evening primrose oil | 3.00 |
| Ethoxylated hydrogenated castor oil (7 EO) | 6.00 |
| Glycerol oleate | 1.00 |
| Phenethyldimethicone | 3.00 |
| Beeswax | 1.50 |
| Carob gum | 0.80 |
| Silk powder | 0.80 |
| Preservatives | q.s. |
| Perfume | q.s. |
| Borax | 0.10 |
| 5′-Deoxy-5′-methylthioadenosine | q.s. |
| Water | 52.30 |

### Example 3

### Multiple Emulsion

| | % by wt. |
|---|---|
| Mineral oil | 7.50 |
| Cetearyl octanoate | 2.50 |
| Aluminum stearate | 0.25 |
| Magnesium stearate | 0.25 |
| Microcrystalline Wax H | 0.50 |
| Cetearyl alcohol | 1.00 |
| Lanolin alcohol | 1.50 |
| Mineral alcohol and lanolin alcohol | 1.50 |
| Ethoxylated hydrogenated castor oil (7 EO) | 0.75 |
| Polyethylene glycol (40 EO)-dodecyl glycol copolymer | 2.00 |
| Ceteareth-6 (reaction product of fatty alcohol | 2.00 |
| and ethylene oxide) and stearyl alcohol | |
| Ceteareth-25 (reaction product of fatty alcohol | 2.00 |
| and ethylene oxide) | |
| Trilaureth-4 phosphate | 1.00 |
| Hydroxyethylcellulose | 0.20 |
| Propylene glycol | 7.50 |
| Magnesium sulfate | 0.25 |
| 5′-Deoxy-5′-methylthioadenosine | q.s. |
| Water | 69.30 |

### Example 4

### Microemulsion

| | % by wt. |
|---|---|
| Ceteareth-25 (reaction product of fatty alcohol | 13.00 |
| and ethylene oxide) | |
| Glyceryl cocoate (7 EO) | 20.00 |
| Octyldodecanol | 5.00 |
| Preservatives | q.s. |
| 5′-Deoxy-5′-methylthioadenosine | q.s. |
| Water | 62.00 |

### Example 5

### Liposome Gel

| | % by wt. |
|---|---|
| Ethoxylated hydrogenated castor oil (40 EO) | 1.00 |
| Bisabolol rac. | 0.10 |
| Propylene glycol | 8.00 |
| Panthenol | 0.50 |
| Water and tocopheryl acetate and polysorbate 80 and | 3.00 |
| caprylic/capric triglyceride and lecithin | |
| Preservatives | q.s. |
| Perfume | q.s. |
| Carbomer | 0.50 |
| 5′-Deoxy-5′-methylthioadenosine | q.s. |
| Triethanolamine | 0.70 |
| Water | 86.30 |

### Example 6

### Blunted Oil Gel

| | % by wt. |
|---|---|
| Silicon dioxide | 5.00 |
| Dimethicone | 10.00 |
| Cetearyl octanoate | 40.00 |
| Caprylic/capric triglyceride | 8.00 |
| Phenethyldimethicone | 2.00 |
| Mineral oil | 28.50 |
| Sweet almond oil | 5.00 |
| Phytantriol | 0.30 |
| 5′-Deoxy-5′-methylthioadenosine | q.s. |
| Tocopherol | 0.50 |
| Perfume | 1.00 |

### Example 7

### Oil Gel

| | % by wt. |
|---|---|
| Silicon dioxide | 5.00 |
| Dimethicone | 10.00 |
| Cetearyl octanoate | 30.00 |
| Caprylic/capric triglyceride | 10.00 |
| Isopropyl myristate | 5.00 |
| Phenethyldimethicone | 5.00 |
| Mineral oil | 28.20 |
| Jojoba oil | 5.00 |
| Phytantriol | 0.30 |
| 5′-Deoxy-5′-methylthioadenosine | q.s. |
| Tocopherol | 0.50 |
| Perfume | 1.00 |

### Example 8

### Cooling Body Splash

| | % by wt. |
|---|---|
| Ethoxylated hydrogenated castor oil (40 EO) | 2.00 |
| Menthyl lactate | 0.20 |
| Alcohol | 5.00 |
| Glyceryl cocoate (7 EO) | 2.00 |
| Witch hazel | 5.00 |
| Allantoin | 0.10 |
| Bisabolol rac. | 0.20 |
| Propylene glycol | 5.00 |
| Panthenol USP | 0.50 |
| Lactic acid (80% strength) | 0.20 |
| 5′-Deoxy-5′-methylthioadenosine | q.s. |
| Perfume | q.s. |
| Water | 79.80 |

### Example 9

### Make-Up

| | % by wt. |
|---|---|
| Ceteareth-6 (reaction product of fatty alcohol | 9.00 |
| and ethylene oxide) and stearyl alcohol | |
| Dimethicone | 5.00 |
| Cetearyl octanoate | 8.00 |
| Macadamia nut oil | 5.00 |
| Propylene glycol | 5.00 |
| Water | 53.00 |
| Sicovit white E 171 | 8.00 |
| Sicomet brown 70 13E 3717 | 2.00 |
| 5′-Deoxy-5′-methylthioadenosine | q.s. |
| Perfume | q.s. |
| 3-Benzophenone | 5.00 |

### Example 10

### Liquid Make-Up

| | % by wt. |
|---|---|
| Ceteareth-6 (reaction product of fatty alcohol | 7.00 |
| and ethylene oxide) and stearyl alcohol | |
| Ceteareth-25 | 5.00 |
| Dimethicone | 5.00 |
| Cetearyl octanoate | 8.00 |
| Macadamia nut oil | 5.00 |
| Propylene glycol | 5.00 |
| Water | 53.00 |
| Sicovit white E 171 | 8.00 |
| Sicomet brown 70 13E 3717 | 1.00 |
| 5′-Deoxy-5′-methylthioadenosine | q.s. |
| Perfume | q.s. |
| 3-Benzophenone | 5.00 |

### Example 11

### Deep Face Cleanser

| | % by wt. |
|---|---|
| Water | 65.60 |
| Cocamidopropylbetaine | 5.00 |
| Sodium cocoate, hydrolyzed animal protein | 8.00 |
| Ethoxylated hydrogenated castor oil (40 EO) | 2.00 |
| Polyquaternium-44 | 7.70 |
| Bisabolol rac. | 0.20 |
| Panthenol | 1.00 |
| Perfume | 0.50 |
| Hydroxyethylcellulose | 2.00 |
| 5′-Deoxy-5′-methylthioadenosine | q.s. |
| Propylene glycol | 5.00 |
| Jojoba wax | 3.00 |

### Example 12

### Conditioner

| | % by wt. |
|---|---|
| Ceteareth-6 and stearyl alcohol | 2.00 |
| Ceteareth-25 (reaction product of fatty alcohol | 1.00 |
| and ethylene oxide) | |
| Cetearyl octanoate | 6.00 |
| Ceteareth-3 | 2.00 |
| Cetearyl alcohol | 6.00 |
| Phytantriol | 1.00 |
| Propylene glycol | 5.00 |
| Polyquaternium-11 | 5.00 |
| Panthenol | 1.00 |
| Retinyl acetate | 0.50 |
| Perfume | q.s. |
| 5′-Deoxy-5′-methylthioadenosine | q.s. |
| Preservatives | q.s. |
| Water | 70.50 |

### Example 13

### Hair Wax

| | % by wt. |
|---|---|
| Polyethylene glycol-6 | 30.00 |
| Polyethylene glycol-75 | 45.00 |
| Liquid paraffin oil | 0.50 |
| Ethoxylated hydrogenated castor oil (40 EO) | 1.00 |
| Glycerol | 15.00 |
| 3-Benzophenone | 2.00 |
| Phytantriol | 0.10 |
| 5′-Deoxy-5′-methylthioadenosine | q.s. |
| Perfume | q.s. |
| Water | 6.40 |

### Example 14

### Antidandruff Hair Tonic

| | % by wt. |
|---|---|
| Alcohol | 45.00 |
| Aloe Vera (10-fold conc.) | 1.00 |
| Panthenol | 1.00 |
| Tocopheryl acetate | 0.50 |
| Ethoxylated hydrogenated castor oil (40 EO) | 0.50 |
| Allantoin | 0.10 |
| Hydrolyzed animal protein | 1.50 |
| 1-(4-Chlorophenoxy)-1-(1H-imidazolyl)-3,3-dimethyl- | 0.30 |
| 2-butanone | |
| Perfume | 0.10 |
| 5′-Deoxy-5′-methylthioadenosine | q.s. |
| Water | 50.00 |

### Example 15

### Foot Deodorant Spray

| | % by wt. |
|---|---|
| Ethoxylated hydrogenated castor oil (40 EO) | 0.80 |
| Alcohol | 20.00 |
| Farnesol | 0.12 |
| Menthyl lactate | 0.08 |
| 1,2 Propylene glycol | 3.20 |
| 4-Benzophenone | 1.20 |
| Glycerol cocoate (7 EO) | 0.80 |
| Perfume | q.s. |
| 5′-Deoxy-5′-methylthioadenosine | q.s. |
| Water | 13.80 |
| Butane | 60.00 |

### Example 16

### Hairspray

| | % by wt. |
|---|---|
| Aminomethylpropanol | 0.40 |
| Dimethicone copolyol | 0.030 |
| Alcohol | 43.67 |
| Pentane | 14.20 |
| Acrylate/acrylamide copolymer | 3.40 |
| Perfume | q.s. |
| 5′-Deoxy-5′-methylthioadenosine | q.s. |
| Butanes | 2.40 |
| Isobutane | 35.90 |

We claim:

1. A method for the cosmetic treatment of the skin in which a cosmetic preparation comprising 5′-deoxy-5′-methylthioadenosine and/or a cosmetically acceptable salt thereof is applied to the skin in an amount effective for improving the appearance of the skin.

2. A method as claimed in claim 1 where the appearance of the skin is changed as a consequence of aging.

3. A method as claimed in either of the preceding claims, where the composition comprises at least one cosmetically acceptable carrier.

4. A method as claimed in any of the preceding claims, where the composition comprises 5′-deoxy-5′-methylthioadenosine in an amount of from 0.001 to 30% by weight based on the total weight of the composition.

5. A skin cosmetic composition, except for compositions for accelerating tanning, comprising
I) 5′-deoxy-5′-methylthioadenosine and/or a cosmetically acceptable salt thereof,
II) optionally at least one further cosmetic active ingredient, and
III)a cosmetically acceptable carrier.

6. A composition as claimed in claim 5 which comprises the component I in an amount of from 0.001 to 30% by weight, based on the total weight of the composition.

7. A composition as claimed in either claim 5 or 6 in the form of a commercial pack, optionally together with instructions for cosmetic application.
